(19) United States
(12) Patent Application Publication
(10) Pub. No.: US 2010/0098652 A1
(43) Pub. Date: Apr. 22, 2010

(54) Compositions and Methods for Treating Hair

(75) Inventors: David S. Soane, Chestnut Hill, MA (US); Michael C. Berg, Baltimore, MD (US); William A. Mowers, Ballston Spa, NY (US)

Correspondence Address: NUTTER MCCLENNEN & FISH LLP, SEAPORT WEST, 155 SEAPORT BOULEVARD, BOSTON, MA, 02210-2604, US

(73) Assignee: Soane Labs, LLC, Cambridge, MA (US)

(21) Appl. No.: 12/469,128

(22) Filed: May 20, 2009

Related U.S. Application Data
(63) Continuation of application No. PCT/US07/24511, filed on Nov. 28, 2007.
(60) Provisional application No. 60/867,447, filed on Nov. 28, 2006.

(51) Int. Cl. A61K 8/898; A61Q 5/12; C08G 77/26

(52) U.S. Cl. 424/70.122; 528/38

(57) ABSTRACT

Compositions and methods for treating hair are disclosed. Such compositions and methods can be used to modify hair properties, such as imparting and/or maintaining a level of softness or a conditioning effect on hair. In general, such compositions can utilize a copolymer having a plurality of polycationic segments, which can be joined by various other polymeric segments such as silicones and/or hydrophilic polymeric segments. The copolymer can be formulated as a highly-branched polymer, which can add substantivity to the hair to impart a conditioning effect. Various details of such copolymers are discussed, as well as methods of utilizing and making such copolymers.

### CROSS REFERENCE TO RELATED APPLICATIONS

This application is a continuation of PCT Application No. PCT/US2007/024511, filed Nov. 28, 2007, entitled “Compositions and Methods for Treating Hair,” which claims the benefit of U.S. Provisional Application bearing Ser. No. 60/867,447 entitled “Compositions and Methods for Treating Hair,” filed on Nov. 28, 2006, the entire contents of both of which are hereby incorporated herein by reference.

The present application is also related to an International Patent Application bearing Application Number PCT/US06/041564, entitled “COMPOSITIONS AND METHODS FOR IMPARTING OIL REPELLENCY AND/OR WATER REPELLENCY,” filed on Oct. 23, 2006. The entire contents of the international application are hereby incorporated by reference herein.

### FIELD OF INVENTION

The technical field of this invention is directed toward compositions that are potentially useful for treating hair, e.g., giving hair a conditioned effect.

### BACKGROUND OF THE INVENTION

Human hair naturally becomes soiled over the course of time due to the sebum that is secreted by the head, as well as soil and other atmospheric contaminants that accumulate on the hair and scalp. Sebum begins to accumulate on the hair extremely fast, leaving the hair with a greasy, dirty feel and at the same time interferes with the control and manageability. It is well known to those skilled in the art that the most effective shampoos for cleansing the hair of the contaminants and sebum are those that contain high lather and synthetic anionic detergents. The process is very efficient at removal of unwanted contamination; however after rinsing with water, the hair is left with what is termed a dry touch or a “creak” in how the hair feels to the touch.

After a thorough cleaning, the hair is left in a state that is difficult to manage and control, due to a tendency for the hair in this state to form kinks, snarls, or physical entanglements with one another. In this state, the hair can become tangled, unmanageable, and difficult to comb in either the dry or wet state. Another undesirable property that arises from this process is that the hair is left in a bare and natural negatively charged state that easily picks up static electricity, thus causing the hair to become even more unmanageable. The hair is composed of amino acids with a distribution that yields a net negative charge.

### SUMMARY OF THE INVENTION

Embodiments are directed to compositions and methods for treating hair. Such compositions and methods can be used to modify hair properties, such as imparting and/or maintaining a level of softness or a conditioning effect on hair. In general, such compositions can utilize a polymer (e.g., a copolymer having a plurality of polycationic segments), which can be joined by various other polymeric segments such as silicones and/or hydrophilic polymeric segments. The polymer can be a highly-branched polymer, which can add substantivity to the hair to impart a conditioning effect.

The negative charge on hair can be used to physically and/or electrostatically attract a conditioning agent to the hair after a shampoo treatment. In general, a conditioning agent can be a cationic entity. Accordingly, in some embodiments of the invention, the cationic component is a polycation. The polymer can be electrostatically attracted to the negatively charged hair and can deposit to form a thin film surrounding the hair. This film can have many beneficial traits. For example, as the polycation deposits on the surface, it can form a uniform coating around the hair. This coating can add weight to the hair, making it more manageable and heavier, simulating a healthier, thicker set of hair while at the same time, the film can impart a smooth surface on all types of hair, including the natural surface morphology as well as that obtained by chemical processing and damage. Smooth surfaces can reflect light yielding a glossier and healthier looking hair. Another benefit could be in UV blockers. By imparting a UV blocker to the hair, the coating can act as protecting agent, which can protect the hair from harmful UV radiation. Another beneficial aspect of this type of conditioner is found in how the hair feels. The addition of silicones to the coating can yield a softer touch to the hair.

Some embodiments of the invention are directed to a copolymer that can be used to treat hair. Such treatment can impart a conditioning effect. The conditioning effect can remain after washing the hair. The copolymers can include polycationic segments, where each of the segments has multiple repeat units that each include at least one amine group. Multiple segments, such as polymeric segments, can be attached to the polycationic segments to form a large copolymer molecule that can be highly-branched, with a high molecular weight (e.g., the copolymer has a molecular weight greater than about 200,000 Daltons). In some embodiments, about 2% to about 40% of the amine groups of a polycationic segment are reacted with another segment, such as a silicone segment (e.g., a silicone segment having multiple functionalities for reacting with amine groups), a hydrophilic segment, or a combination of silicone and hydrophilic segments.

In some embodiments, the copolymer includes a plurality of silicone segments. The silicone segments can be attached to one, two, or more distinct polycationic segments. The copolymer can also, or alternatively, include a plurality of hydrophilic segments, where each hydrophilic segment can be attached to one, two, or more distinct polycationic segments. To allow a silicone segment or a hydrophilic segment to attach to a polycationic segment, the silicone segment or hydrophilic segment can be functionalized (e.g., at a terminal end) to allow reaction with an amine group of the polycationic segment.

Polycationic segments that are utilized in copolymer embodiments include aliphatic amine polymer segments, which can include at least one of polyalkyleneimine, polyvinylamine, polyallylamine, polydiallylamine, and copolymers thereof. For example, a polyalkyleneimine segment that is used as part of the copolymer can comprise a repeat unit represented by Structural Formula (Ia):

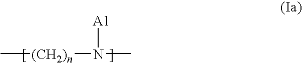

wherein n is an integer from about 2 to about 10. A1 can be at least one of a hydrogen atom, a silicone segment, and a hydrophilic segment. In some embodiments, n is from about 2 to about 5. In other embodiments, the polyalkyleneimine segment is a polyethyleneimine segment. The polycationic segments can be linear or branched. Branched polycationic segments, such as aliphatic amine polymer segments, can have a degree of branching below a designated value such as about 0.33. The polycationic segments can have an average molecular weight greater than 100,000 Daltons, or the average molecular weight can be in a range between about 10,000 Daltons and 2,000,000 Daltons, or the average molecular weight can be in a range between about 100,000 Daltons and 500,000 Daltons.

Different types of silicone segments can be utilized with the various copolymers consistent with embodiments of the invention. For example, one or more silicone segments can include a polymer segment having a repeat unit represented by Structural Formula (II):

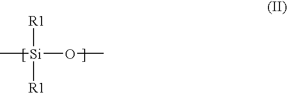

where each R1 group is independently a hydrocarbyl group containing 1 to 6 carbon atoms, a hydrogen, or a hydroxyl group. In another embodiment, the hydrocarbyl group of R1 can be at least one of an alkyl group with 1 to 4 carbons, phenyl, and vinyl. In another embodiment, each R1 group is independently at least one of hydrogen, phenyl, and methyl. In some embodiments, the silicone segments have an average molecular weight between about 500 Daltons and 10,000 Daltons, or between about 500 Daltons and about 2,000 Daltons. Some embodiments include a plurality of softening segments that are each functionalized to attach to only one polycationic segment. Such single point attached softening segments can impart a softness to the hair, when the copolymer is applied thereto. In some embodiments, the softening segments include single point attached silicone segments. Such silicone segments can have an average molecular weight greater than about 3,000 Daltons.

Hydrophilic segments that can be utilized with embodiments of the invention include a variety of polymeric segments that can be substantially non-ionic. For instance, the hydrophilic segment can include segments from a monomer comprising at least one of a sugar and a hydroxide group. In some embodiments, the hydrophilic segments include a polymeric segment having a repeat unit of a polyalkylene oxide, such as a polyethylene oxide or polypropylene oxide repeat unit. Hydrophilic segments, such as polyalkylene oxides, can have an average molecular weight between about 300 Daltons and about 100,000 Daltons, or between about 500 Daltons, and about 5,000 Daltons.

Other embodiments of the invention are directed to hair-treating compositions. The compositions can include a polymer mixture comprising one or more of the copolymers disclosed in the present application. Such compositions can be formulated as a hair treating composition (e.g., a conditioner or 2-in-1 shampoo/conditioner composition). The polymer mixture can include an aqueous solvent, a non-aqueous solvent, or a mixture of aqueous and non-aqueous solvents. In some embodiments, such treatment solutions can include one or more other components such as a UV blocker, a dye, an optical brightening agent, a thickener, a deposition agent, a cleansing agent (e.g., a shampooing agent), a hindered amine light stabilizer, or a fragrance material. These other components can be separate components from the copolymer, or one or more of the components can bind to a section of the copolymer. For example, a polycationic segment of a copolymer can include a repeat unit having Structural Formula (Ib):

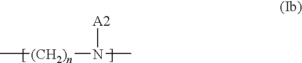

wherein n can be any of the values disclosed for Structural Formula (Ia); and A2 can be at least one of a UV blocker, a dye, an optical brightening agent, a thickener, a deposition agent, a hindered amine light stabilizer, and a fragrance material. Treating compositions can generally include the copolymer in a concentration range from about 1 part per million to about 40% by weight of the entire mixture.

The copolymer can be applied in a variety of manners. In some embodiments, a polymer mixture, which can be aqueous, non-aqueous, or a combination thereof, is formed with the copolymer. The polymer mixture is contacted with the hair to apply the copolymer thereto. In other embodiments, the copolymer is applied by forming the copolymer in the vicinity of the hair. Polycations can be applied to the hair that can have properties consistent with polycationic segments of the copolymer (e.g., a branched polycation such as polyethyleneimine). Subsequently, a mixture including silicone polymers can be introduced, where the silicone polymers can have one or more functionalities for binding to an amine group of one or more polycations. The silicone polymer can be reacted with an amine group to form the copolymer. For example, heat can be introduced to drive the reaction of the amine group with a polymer having a functionality to induce binding. A hydrophilic polymer, consistent with one or more of the properties of hydrophilic segments disclosed herein, can also, or alternatively, be reacted with an amine group of a polycation to cause attachment.

### DETAILED DESCRIPTION OF EMBODIMENTS OF THE INVENTION

Embodiments of the invention are directed to methods and compositions for enhancing the softness of hair giving it a conditioning effect. Some embodiments include the use of a polymer that can be exposed to hair to augment its softness. Such embodiments include polymers that are dispersible in an aqueous solution (e.g., the polymers do not form an emulsion). Other polymers, in accord with embodiments of the invention, can be delivered in a non-aqueous solution or a mixture of aqueous and non-aqueous solutions. As well, in some embodiments, the compositions can be easily synthesized using commercially-available starting materials, avoiding excessive costs of requiring expensive chemical precursors and/or manufacturing equipment.

As utilized within the present application, the term “polymer” refers to a molecule comprising a plurality of repeat units or monomers. A polymer can comprise one or more distinct repeat units. For example, a “copolymer” refers to a polymer having two or more distinct repeat units. Repeat units can be arranged in a variety of manners. For example, a homopolymer refers to a polymer with one type of repeat unit where the repeat units are adjacently connected. In another example, a plurality of different repeat units can be assembled as a copolymer. If A represents one repeat unit and B represents another repeat unit, copolymers can be represented as blocks of joined units (e.g., A-A-A-A-A-A . . . B-B-B-B-B-B . . . ) or interstitially spaced units (e.g., A-B-A-B-A-B . . . or A-A-B-A-A-B-A-A-B . . . ), or randomly arranged units. Of course, these representations can be made with 3 or more types of repeat units as well. In general, polymers (e.g., homopolymers or copolymers) include macromolecules in a broad range of configurations (e.g., cross-linked, linear, and/or branched). A “highly-branched polymer” refers to a branched and/or cross-linked polymer where the molecule has a tendency to form a three-dimensional space filling structure. For example, a highly-branched polymer can have a configuration where the ratio of the number of branches with each of its ends connected to cross-linkages and/or branch points to the number of branches having a free end is greater than some designated value (e.g., greater than about 0.5, 0.6, 0.7, 0.8, 0.9, 1.0, 1.50, 2.0, or 5.0). The polymer can be disposed in a variety of mixture dispositions such as solutions, melts, and/or gels. A gel refers to a state where a mixture of polymer and liquid has at least some properties that make the mixture behave more like a solid than a viscous liquid (e.g., the mixture exhibits elasticity). Various embodiments described herein are directed to compositions, or use thereof, that include a polymer having one or more of the specific properties discussed above.

As utilized in the present application, the term “segment” refers to a portion of a copolymer molecule. In many instances, the segments can be polymeric segments (i.e., portions or the entirety of a polymer) that are reacted with other segments to form the copolymer molecule.

### Copolymers for Treating Hair

Some illustrative embodiments are directed to a copolymer that can be applied to hair. Such copolymers can impart and/or maintain a degree of conditioning or softness in the hair. The copolymer can comprise a plurality of polycationic segments. Each polycationic segment can be at least a portion of a polymer having a plurality of repeat units, with each repeat unit including at least one amine group. It is understood, however, that a multiplicity of amine groups associated with a repeat unit can also be used. Polycationic segments can be bound to a plurality of other types of segments, as revealed in the present application, to form the copolymer.

In some embodiments, the copolymer can comprise a plurality of polycationic segments, in various forms as disclosed throughout the present application, and a plurality of silicone segments, such as a plurality of portions of one or more types of silicone polymers. Each silicone segment can be attached (e.g., bonded) to at least one polycationic segment. For example, each of the silicon segments can be attached to a polycationic segment at an amine group of the polycationic segment.

In some particular embodiments, each silicone segment can be attached to at least two distinct polycationic segments (i.e., each polycationic segment has a distinct backbone relative the other polycationic segments). The latter embodiments can promote the formation of a highly-branched copolymer molecule, with the silicone segments acting as hydrophobic connectors between the polycationic segments. Though the copolymer can take on any molecular weight value, in some embodiments the average molecular weight of the copolymer can be large when the copolymer forms a highly-branched structure. For example, the average molecular weight of a copolymer can be greater than about 200,000 Daltons, greater than about 750,000 Daltons, greater than about 1,000,000 Daltons, or greater than about 3,000,000 Daltons. Without necessarily being bound by any particular theory, it is believed that larger molecular weight copolymers can generally provide better substantivity to the hair relative to known conditioning compositions for treating hair.

Measurement of the average molecular weights for any polymer discussed herein can be with respect to a number of bases. For example, can be number averaged, weight averaged, or averaged based on some other weighting factors. As well, the techniques utilized to determine molecular weight can include the range of those known to those skilled in the art. Examples include gel permeation chromatography and light-scattering.

Without necessarily being bound by theory, it is believed that when hairs are contacted with a copolymer mixture, consistent with embodiments described herein, the polycationic segments tend to assemble onto the hair surface through electrostatic interactions. For example, when the polycationic segment includes a portion of a polyalkyleneimine, the residual charge density of the amine groups on the backbone are conjectured to interact with the hair surface, and induce assemblage thereon. In some instances, it is believed that the polycationic segments can form crystalline-like domains, which can substantially improve the affinity between the copolymer and a hair surface. Furthermore, it is believed that copolymers can be in the form of a highly-branched copolymer molecule, which can be readily deposited from a copolymer mixture to a hair surface, thereby providing additional stability/affinity for the copolymer on the surface. Accordingly, some embodiments that include the highly branched copolymer molecule can form a conditioner that allows hair to be exposed to multiple washings without losing the conditioning effect imparted by the copolymer. As well, embodiments can also include the ability to maintain any of the advantageous properties discussed herein with respect to hair treatment after one or more washings after the copolymer is applied.

Again, without limiting the scope of the present invention, it is believed that the silicone segments can impart desirable softness and to some extent water repellency and/or oil repellency properties. As well, it is believed that the silicone segments can improve the copolymer's tendency to exhaust from a mixture (i.e., lower concentrations of polymer in the polymer mixture can be utilized to treat hair when the polymer is soluble in the mixture but close to coming out of solution).

A variety of polycationic segments can be utilized with various embodiments that include one or more of the copolymers described herein. Polycationic segments can be naturally occurring macromolecules with amine groups such as chitosan, or various types of synthetic polymers (e.g., copolymers) bearing amine groups. In some embodiments, the plurality of polycationic segments can include one or more aliphatic amine polymer segments. Aliphatic amine polymers include aliphatic polymers having one or more amine groups in each of a repeat unit of the polymer. Non-limiting examples of aliphatic amine polymers include polyalkyleneimine, polyvinylamine, polyallylamine, and polydiallylamine. Aliphatic amine polymers can also include copolymers having repeat units of different types of aliphatic amine homopolymers, such as copolymer utilizing repeat units of the examples of aliphatic amine polymers.

Consistent with embodiments disclosed herein, polycationic species bearing a multiplicity of secondary amines (e.g., polyalkyleneimines) can be reacted with other segments at the secondary amine locations to connect distinct polycationic segments. This can help promote formation of a highly-branched copolymer molecule. It is understood, however, that some segments, such as silicone segments and hydrophilic segments, can form loops along a single polycationic segment of the copolymers as well. Though the presence of secondary amines can promote the formation of copolymers consistent with embodiments disclosed herein, it is generally understood that the amine groups of a polycationic segment can include primary, secondary, tertiary, or quaternary amines. For example, the presence of some quaternary amine groups can help promote dispersion of a copolymer in an aqueous solvent.

For embodiments utilizing a polyalkyleneimine segment, the segment can include a repeat unit having Structural Formula (Ia):

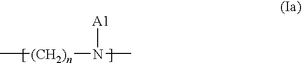

where n is an integer from about 2 to about 10. Alternatively, n can be an integer from about 2 to about 5. In another alternative, Structural Formula (Ia) is a repeat unit for a polyethyleneimine (i.e., n is 2). A1 can be at least one of a hydrogen atom, a silicone segment, a hydrophilic segment, a UV blocker, a dye, an optical brightening agent, a thickener, a deposition agent, a hindered amine light stabilizer, and a fragrance material. In a particular embodiment, A1 can be at least one of a hydrogen atom, a silicone segment, and a hydrophilic segment. In another particular embodiment, A1 can be at least one of a hydrogen atom and a silicone segment.

Polycationic segments employed with various embodiments can have a variety of molecular weights and molecular weight ranges. In general, a desirable molecular weight range for the polycationic segments is large enough to promote branch formation of the copolymer and small enough such that the polycationic segment can be dispersed in a solvent without undue effort. For example, in some embodiments, the polycationic segments (e.g., polyalkyleneimines such as polyethyleneimine) can have an average molecular weight greater than about 100,000 Daltons. In some other embodiments, the polycationic segments (e.g., polyalkyleneimines such as polyethyleneimine) have an average molecular weight between about 10,000 Daltons and about 2,000,000 Daltons, or between about 100,000 Daltons and about 500,000 Daltons.

Generally, polycationic segments (e.g., polyalkyleneimine segments such as polyethyleneimine) can be either linear or branched to various degrees. In some embodiments, the polycationic segment has a degree of branching in a particular range. The degree of branching along a linear backbone portion of a polymer is defined herein as the number of branching side chains coming off that linear backbone portion divided by the total number of possible branches that can potentially come off that linear backbone portion. Accordingly, the degree of branching is a value between 0 and 1, with 0 corresponding to the portion being completely linear and 1 corresponding to the portion being completely substituted with side branches. Herein, the degree of branching can be with respect to any chosen linear portion of a polymer segment (e.g., the longest linear portion of a branched polymer segment). As well, when referring to the degree of branching off a polymer segment herein, the branching is measured with respect to the number of branches that are of the same character as the polymer segment. For example, in calculating the degree of branching of a polycationic segment, side chains connected to the segment that are of a different chemical nature, such as a silicone segment or a hydrophilic segment, are not considered.

In some embodiments, copolymers having polycationic segments with low degrees of branching are employed. For example, a plurality of the polycationic segments (e.g., polyalkyleneimines such as polyethyleneimine) have a degree of branching lower than about 0.33, or lower than about 0.10, or lower than about 0.05, or lower than about 0.01. In one embodiment, the polycationic segment is approximately or substantially linear, i.e., the degree of branching approaches zero. The degree of branching can be with respect to any linear branch of a branched polycationic segment such as the longest linear branch. Without being bound by theory, it is believed that polycationic segments that are more linear can improve the affinity of the copolymer for a hair surface by promoting the tendency for the copolymer to form crystalline domains. As well, more branched polycationic segments can result in segment conformations that decrease the accessibility of silicone segments, and other copolymer components, to reach amine groups of the polycationic segment for reaction and binding. As a result, the overall branching of the copolymer can be decreased, resulting in potentially less affinity and/or stability of the copolymer with a hair surface when the formed copolymer is delivered as a mixture to the hair surface.

Within the scope of some embodiments of the invention, different types of silicone segments can be utilized with the copolymers discussed herein. In some embodiments, a silicone segment can be a polymeric segment. Such polymeric segments can include a repeat unit represented by Structural Formula (II):

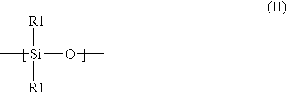

where each R1 in Structural Formula (II) is independently a substituted or unsubstituted hydrocarbyl group, a hydrogen, or a hydroxyl group. Hydrocarbyl groups that can be utilized include both aliphatic and aromatic groups that can be optionally substituted with another aliphatic functionality and/or a heteroatom functionality (e.g., any combination of sulfur, oxygen, or nitrogen). Hydrocarbyl groups can include any number of carbon atoms such as 1 to 10 carbon atoms. Non-limiting examples of hydrocarbyl groups include a vinyl group; a substituted or unsubstituted phenyl group, such as unsubstituted phenyl and phenyl substituted at one or more positions with methyl, ethyl, or propyl; and substituted or unsubstituted alkyl groups, such as alkyl groups with 1 to 4 carbons, or more particularly methyl or ethyl. Accordingly, in some embodiments, each R1 can independently be a hydrocarbyl group containing 1 to 10 carbon atoms, a hydrogen, or a hydroxyl group; or each R1 can independently be an alkyl group with 1 to 4 carbon atoms, phenyl, vinyl, or hydrogen; or each R1 can independently be hydrogen, phenyl, or methyl. In a particular embodiment, the silicone segments can include one or more polydimethylsiloxane (“PDMS”) segments.

Generally, the silicone segments can include one or more functional groups for reacting with a portion of a polycationic segment to produce attachment during copolymer synthesis. Such functional group(s) can be located at a terminal end of a silicone segment, or in the neighborhood of a terminal end, or anywhere within the silicone segment. In one embodiment, a silicone segment can include an amine-reacting functionality at each of two terminal ends of the silicone segment. Such a segment can be used to attach each of the functionalized ends to a distinct polycationic segment, which can be beneficial for forming a highly-branched copolymer molecule. Other silicone segment embodiments can utilize three or more functional groups such that a silicone segment can bind in more than two places with one or more polycationic segments.

The chemical nature of the functional group of a silicone segment can be selected to allow reaction between the functional group and a site on a polycationic segment. In some embodiments, the functional group is selected to allow reaction with an amine group of a polycationic segment. Non-limiting examples of such functional groups include epoxides, isocynates, alkyl halides (e.g., methylchloryls), anhydrides, and other amine-reacting functional groups known to those skilled in the art.

The silicone segments, which can be used with copolymers consistent with embodiments revealed in the present application, can span a variety of sizes and structures. For instance, the silicone segments can be branched or linear, and can have a variety of molecular weights. In general, the molecular weight of the silicone segments can be selected to alter the end properties of the copolymer (e.g., ability to exhaust from a mixture; ability to impart softness). In some embodiments, the average molecular weight of the silicone segments (e.g., PDMS segments) is between about 500 Daltons and about 10,000 Daltons, or between about 500 Daltons and 2,000 Daltons.

In some particular embodiments, the copolymer includes a plurality of monofunctionalized softening segments, which can be attached at the functionalization location to a polycationic segment, for instance at an amine group of the polycationic segment. As an example, a copolymer can include both multifunctionalized silicone segments and monofunctionalized silicone segments attached to one or more polycationic segments. The presence of the monofunctionalized softening segments can help impart an enhanced “softness” quality to treated hair. Softening segments include polymeric segments that have a low T_{g} (e.g., polymeric segments with a T_{g} lower than about 30° C., or lower than the polycationic segments). Examples of softening segments include rubbers such as polyisoprene, and monofunctionalized silicone segments. Types of silicone segments and functionalities that can be used in these embodiments include the silicone segments and functionalities previously described. In one particular embodiment, the silicone segments (e.g., PDMS segments) utilized have an average molecular weight greater than about 3,000 Daltons.

For the various copolymers revealed within the present application, embodiments including the copolymer can comprise a plurality of hydrophilic segments. For example, the copolymer can comprise a plurality of polycationic segments, which can have any of the properties of the polycationic segments revealed in the present application, along with the hydrophilic segments. In a particular embodiment, the copolymer comprises a plurality of polycationic segments and multiple silicone segments, the segments having any of the properties revealed in the present application, and the plurality of hydrophilic segments. Each hydrophilic segment can be attached to one, two, or more distinct polycationic segments, for example at an amine group of the polycationic segment. When the hydrophilic segment is selected to allow attachment to two or more distinct polycationic segments, the hydrophilic segment can aid in branching of the copolymer. Accordingly, the average molecular weight of such copolymers can be greater than about 200,000 Daltons, about 750,000 Daltons, about 1,000,000 Daltons, or about 3,000,000 Daltons. Without necessarily being bound to any particular theory, it is believed that the presence of the hydrophilic segments can aid in the solubility and dispersibility of the copolymer in aqueous solutions, providing potentially better dispersibility of the composition relative to other known agents.

The types of hydrophilic segments that can be utilized include, typically, polymeric segments that enhance the dispersibility of the copolymer in aqueous mixtures. For instance, the hydrophilic segments can be substantially non-ionic, having limited charge or substantially no charge, such as to limit interference with the polycationic segments. Non-limiting examples of hydrophilic segments include sugar-based polymers such as hydroxypropyl cellulose, dextran polymers and their derivatives, and other polysaccharides or carbohydrates; polyethers such as polyalkylene oxides like polypropylene oxide and polyethylene oxide; polymers having hydroxide containing repeat units such as polyvinyl alcohol; polyvinyl pyrrolidone; and other polymer segments with the desired properties as known to those skilled in the art. In some embodiments, hydrophilic segments can include copolymers with one or more repeat units that are utilized in a sugar-based polymer and/or a polyether.

In some embodiments, the hydrophilic segments include repeat units from a polyether, or the segments are substantially one or more polyether segments. In some particular embodiments, the polyethers include a polyalkylene oxide based polymer. Such a polymer can include repeat units represented by Structural Formula (III):

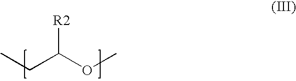

where each R2 is independently a hydrogen, or a substituted or unsubstituted aliphatic group. Potential substitutions include another aliphatic functionality and/or a heteroatom functionality. In some embodiments, each R2 is independently a hydrogen, or an optionally substituted saturated aliphatic group with 1 to 6 carbon atoms, or 1 to 3 carbon atoms. In one particular embodiment, each R2 is independently a hydrogen or a methyl. For example, the hydrophilic segment can comprise a polyethylene oxide segment, a polypropylene oxide segment, or a copolymer having a mixture of both segments.

Generally, the hydrophilic segments can include one or more functional groups for reacting with a portion of a polycationic segment to produce attachment during copolymer synthesis. Such functional group(s) can be located at a terminal end of a hydrophilic segment, or in the neighborhood of a terminal end, or anywhere within the hydrophilic segment. In one embodiment, the functionality is an amine-reacting functionality at one or two terminal ends of the hydrophilic segment. Hydrophilic segments with two amine-connecting functionalities can be used to attach each of the functionalized locations to two distinct polycationic segments, which can enhance copolymer branching. Of course, more than two functionalities per hydrophilic segment can be used.

Functionalizations for hydrophilic segments typically are selected to allow attachment of the hydrophilic segment to a polycationic segment (e.g., an amine-reacting functionalization). Non-limiting examples of such functional groups include epoxides, isocynates, alkyl halides (e.g., methylchloryls), anhydrides, and other amine-reacting functional groups known to those skilled in the art.

Hydrophilic segments embodied as polymer segments can include polymer segments that are linear or branched to various degrees. Hydrophilic segments can also have a variety of molecular weights. In some embodiments, the molecular weights are selected to promote copolymer solubility in aqueous mixtures. For example, hydrophilic segments (e.g., polypropylene oxide or polyethylene oxide) can have an average molecular weight between about 300 Daltons and 100,000 Daltons, and between about 500 Daltons and about 5,000 Daltons.

Copolymers can be formulated such that a proportion of amine groups associated with a plurality of polycationic segments are bound to selected substituents. Though the proportion of amine groups that are bound to selected substituents can be any percentage, in some embodiments the proportion of amine groups reacted are sufficient such that the copolymer can provide softness and an affinity to the hair. For example, the proportion of amine groups can be greater than about 2%, 5%, 10%, 15%, 20%, 25%, 30%, 35%, 40%, or 50% of all amine groups of the polycationic segments. In particular embodiments, the proportion of selectively bound amine groups of the polycationic segments (e.g., polycationic segments with a particular degree of branching such as less than about 0.33, or other ranges disclosed herein) can be between about 2% and about 40%, or between about 3% and about 35%, of all the amine groups of the polycationic segments. Selected substituents can include a variety of substituents. In some embodiments, the selected substituents are silicone segments, hydrophilic segments, other substituents as described herein (e.g., UV blockers, dyes, deposition agents, etc.) and/or known to those skilled in the art, or any combination of such substituents. In some particular embodiments, the selected substituents include at least one of silicone segments and hydrophilic segments.

Some of the copolymers described herein can be formed from a single step synthesis. For example, polycationic polymers corresponding to the polycationic segments can be mixed with silicone polymers corresponding to the silicone segments in a single vessel to form the copolymer molecules. Ins some instances, some or all of the precursors are commercially available materials. In addition, other components such hydrophilic polymers corresponding to any of the hydrophilic segments disclosed herein and/or UV blockers, dyes, and other materials can also be added to a single vessel. Accordingly, embodiments of the invention can include a mixture of the precursors that will eventually form any of the copolymers of the present application, or intermediate copolymers thereof. Alternatively, separate steps can also be utilized if desired. Those skilled in the art will readily appreciate modifications in these steps to form the copolymers of the present application. Such modifications are all within the scope of the present application.

### Copolymer Dispersion Properties

In accord with embodiments of the invention disclosed herein, the copolymers discussed herein can be formulated to be dispersible in an aqueous solution, a non-aqueous solution, or a mixture of aqueous and non-aqueous solutions. For example, copolymers having a plurality of polycationic segments and silicone segments can be dispersible in aqueous solutions, non-aqueous solutions, or a combination of both. In some embodiments, such copolymers do not require the presence of a hydrophilic segment for dispersibility in non-aqueous solutions or solutions formed from a mixture of aqueous and non-aqueous dispersions (e.g., aqueous solution with isopropyl alcohol). These copolymers can also be dispersed in an aqueous solution which has been acidified to add charge on the polymer and enhance aqueous solution dispersibility. For example, a copolymer solution with less than about 2% (w/v) of copolymer can be acidified to a level with a pH less than about 9 to allow dispersal of a copolymer having substantially linear polycationic segments such as polyethyleneimine. Copolymers with a plurality of polycationic segments, silicone segments, and hydrophilic segments can be dispersed in aqueous solutions. Without being bound by theory, it is believed that the presence of hydrophilic segments can help improve the copolymer's dispersibility in aqueous environments.

Though polymer mixtures having any of the copolymers disclosed herein can have a variety of concentrations and concentration ranges, in some embodiments a range of concentration is selected to allow the copolymer to be dispersed in a dispersion medium. For example, when the copolymer can be stored as a gelled material in some instances, it can be advantageous to limit the concentration of the copolymer in a dispersion medium such that dilution of the copolymer to form a non-gelled solution can be performed without substantial undue effort. In some examples, the polymer mixture can have a concentration of copolymer less than about 50%, or less than about 40%, or less than about 30% by weight. As well, the concentration range of a mixture can be chosen to provide sufficient copolymer to allow the copolymer to be applied to hair and impart or maintain a property (e.g., softness). The polymer mixture can have a concentration of copolymer greater than about 1 part per million, or greater than about 1 part per 100,000, or greater than about 1 part per 1,000 by weight. In some embodiments, the concentration of copolymer in a polymer mixture can be in a range between about 1 part per million and about 50% by weight, or between about 1 part per million and about 40% by weight.

In some embodiments, a polymer mixture having a copolymer, as disclosed herein, is prepared such that the copolymer does not form a gel in the mixture. Such non-gelled polymer solutions can be utilized to apply the copolymer to hair by contacting the solution to the hair; the solid-like properties of gelled materials can substantially hinder copolymer delivery to the hair. In some embodiments, the polymer mixture can have a concentration such that the copolymer does not form a gel, and can exhaust from the non-gelled solution readily for application to hair. For instance, the concentration of copolymer can be just below the gelation point (e.g., less than about 5%, or less than about 2%, or less than about 1% by weight of the solution). In some embodiments, the concentration of a copolymer (e.g., having polyethyleneimine segments connected by silicone segments and polyalkylene oxide segments) is about 0.5% to about 2% by weight of the solution.

The various copolymers disclosed herein can be utilized with other components to provide a treatment composition for hair. Such a treatment can impart and/or maintain a level of softness to the hair. The hair treatment composition can additionally be a hair conditioner, a 2-in-1 shampoo and conditioner, or other hair treatment composition. In such a capacity, the hair treatment composition can include one or more additional components to impart additional properties to treated hair or the composition. Such components can include UV blockers, dyes, optical brightening agents, thickeners, deposition agents, hair cleansing agents, hindered amine light stabilizers, and fragrance materials. For instance, a 2-in-1 shampoo and conditioner would include a cleansing agent as a component, such as various types of surfactants. These components include the range of such materials as known to those skilled in the art. Types of UV blockers that can be utilized include cinnamic acid, vanillin, benzophenone, benzotraizole, and hydroxyphynyl triazine. Types of dyes include textile dyes, food dyes, reactive dyes, and those typically used in formulations known to the skilled artisan. Optical brightening agents, such as fluorescent whitening agents, are commercially available from manufacturers (e.g., Ciba®, Uvitex®, OB, and Durawhite from Town End). Thickeners that can be utilized include polymers such as cellulosics and/or polysaccharides. Deposition aids generally include materials which can aid in putting the copolymer on hair (e.g., other polycations). Fragrances that can be used include all those compatible with hair treatment products. In some embodiments, components can be added to the treatment composition as separate components that are not directly bonded to the copolymer.

Any one of the additional components can be attached to the copolymer directly, which can potentially aid in the component's delivery and/or substantivity to the hair. In some embodiments, one or more of the additional components are bound to a portion of the polycationic segment such as an amine group of a polycationic segment. In some particular embodiments, the polycationic segments can include a repeat unit having Structural Formula (Ib):

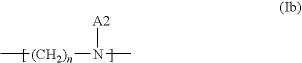

where n is an integer between about 2 and about 10; or between about 2 and about 5; or n is 2. A2 can be an additional component that enhances a treatment composition without significantly affecting the softness or substantivity of the copolymer. In some embodiments, A2 can be at least one of a UV blocker, a dye, an optical brightening agent, a thickener, a deposition agent, a hindered amine light stabilizer, and a fragrance material. In other embodiments, A2 is at least one of a UV blocker, a dye, a thickener, and a deposition agent.

### Methods for Treating Hair

Some embodiments are directed to methods of using any of the copolymers revealed within the present application. For instance, one embodiment is generally directed to a method of treating hair. Such a method includes a step of applying a copolymer to hair to impart softness. The copolymers that can be utilized include one or more of the copolymers disclosed in the present application. For example, the copolymer can comprise a plurality of polycationic segments and silicone segments, or a plurality of polycationic segments, silicone segments, and hydrophilic segments as described within the present application. Such an embodiment can provide any number of properties to hair as discussed in the present application, e.g., imparting softness to hair.

Application of the copolymer can be performed in a variety of manners. In some embodiments, the copolymer can be a portion of a polymer mixture such as a non-gelled polymer solution. The dispersion media can be aqueous (e.g., acidified aqueous), non-aqueous, or a mixture of both. The hair can be contacted with the polymer mixture (e.g., immersion, spraying, or other technique of solution/substrate contact), which can apply the copolymer to the hair. Such mixtures can have any of the properties disclosed for copolymer mixtures herein. The copolymer can adhere to the hair through a variety of interactions such as electrostatic attraction. In the latter instance, a crosslinking agent can be added to the polymer solution, or applied to the hair after contacting the polymer solution with hair, to initiate bonding with the copolymer.

In other embodiments, the copolymer is applied to a hair by forming the copolymer in the presence of the hair. For instance, a plurality of polycations can be applied to the hair. Such polycations can include any of the polycations disclosed herein for use with the copolymers. A mixture of silicone polymers can be subsequently introduced. The silicone polymers can have any of the properties disclosed for silicone segments herein (e.g., each silicone polymer having one, two, or more functionalities for reacting with an amine group to attach to a polycation). Each silicone polymer can react and attach to a polycation to help form the copolymer. In some cases, heat can be applied to induce the copolymer formation (e.g., heating one or more of the mixtures and/or the hair).

Copolymer formation in the vicinity of hair can be associated with potential advantages. For instance, forming a branched copolymer on the surface of the hair can help alleviate the labor associated with processing highly-branched, high molecular weight polymer mixtures, which can have a tendency to gel. Polymer solutions with the polycations, and/or polymer solutions with the silicones can potentially be easier to handle, and may avoid the complications of needing to utilize a polymer solution that is close to its gel point. In another instance, copolymer formation in the vicinity of hair can ease the use of particular copolymer compositions. For example, copolymers that include branched polycationic segments can be difficult to apply as the copolymer molecule to hair due to effects such as potential decreased affinity relative to using more linear polycationic segments in a formed copolymer. By applying the branched polycations first (e.g., branched polyethyleneimine) and then reacting with other segments to form the copolymer, the application of such copolymers can be performed more readily.

Potentially, any of the copolymers disclosed in the present application can be formed in the presence of hair. For example, the copolymer can also include hydrophilic segments, such as any of the types disclosed herein (e.g., with amine reacting functionalities). The hydrophilic segments can be formed from corresponding hydrophilic polymers that can be part of the silicone polymer mixture, or applied as a separate mixture. As well, other components such as UV blockers, optical enhancing agents, thickeners, etc., can be added (e.g., with any of the previously mentioned mixtures, or as one or more other separate mixtures).

Those skilled in the art will readily appreciate that any number of additional steps, and the order in which the steps are carried out, can be modified within the scope of the methods disclosed herein. All such alterations are within the scope of the present application.

### EXAMPLES

The following examples are provided to illustrate some aspects of the present application. The examples, however, are not meant to limit the practice of any embodiment of the invention.

### Synthetic Procedures

### Synthesis 1: “Linear” Polyethyleneimine (“LPEI”)

Poly(2-ethyl-2-oxazoline) (Sigma Aldrich, St. Louis, Mo.; catalog no. 373974) was deacetylated by refluxing in 6M HCl overnight to yield a LPEI with a molecular weight of about 215,000 Daltons.

### Synthesis 2: LPEI/PDMS Copolymer

Poly(dimethylsiloxane) (“PDMS”), having a molecular weight of about 980 Daltons and being diglycidyl ether terminated (Sigmal Aldrich, St. Louis, Mo.; catalog no. 480282), was mixed with LPEI, produced using Synthesis 1, in a thick-walled glass flask with isopropyl alcohol (“IPA”). The amounts of each component for making Sample A1 are shown in Table 1. The flask was heated to 150° C. and kept at that temperature for 18 hours. The resulting polymer solution was then concentrated to 20% (w/v) using a Roto-Vap.

TABLE 1

| Sample | Si1KDi (g) | LPEI (g) | IPA (mL) |
|---|---|---|---|
| A1 | 4.20 | 1.80 | 200.0 |

### Synthesis 3: LPEI/PPO/PDMS Copolymer

Sample B1 of LPEI/PPO/PDMS copolymer were prepared from starting materials as listed in Table 2. For the sample, LPEI and PDMS, as described for Synthesis 2, were mixed with poly(propylene glycol) (“PPO”), having a molecular weight of about 640 Daltons and being diglycidyl ether terminated (Sigma Aldrich, St. Louis, Mo.; catalog no. 406740), in a thick-walled glass flask with IPA. The flask was heated to 150° C. and kept at that temperature for 18 hours. The resulting polymer solution was then concentrated to 20% (w/v) using a Roto-Vap.

TABLE 2

| | LPEI | Si1KDi | PPO | IPA |
|---|---|---|---|---|
| Sample | (g) | (g) | (g) | (mL) |
| B1 | 1.8 | 4.0 | 0.20 | 200.0 |

### Synthesis 4: LPEI//Bifunctional and Monofunctional Silicone Copolymer

Copolymer samples C1-C4 were produced from starting materials as listed in Table 3. For each sample, LPEI and PDMS, as described in Synthesis 3, were mixed with two other types of PDMS: an epoxypropoxypropyl, mono-terminated PDMS having a molecular weight of about 5,000 Daltons (Gelest, Morrisville, Pa.; #DMS-E21); and a chloromethyl, di-terminated PDMS having a molecular weight of about 7,000 Daltons (Gelest, Morrisville, Pa.; #DMS-L21). The components were combined with IPA in a thick-walled glass flask that was heated to 150° C. and kept at that temperature for 18 hours. The resulting polymer solution was then concentrated to 20% (w/v) using a Roto-Vap.

TABLE 3

| | Si5K | Si1KDi | Si7KDi | LPEI | IPA |
|---|---|---|---|---|---|
| Sample | (g) | (g) | (g) | (g) | (mL) |
| C1 | 0.00 | 2.00 | 2.00 | 2.00 | 200.0 |
| C2 | 2.00 | 0.00 | 2.00 | 2.00 | 200.0 |
| C3 | 2.00 | 1.00 | 1.00 | 2.00 | 200.0 |
| C4 | 1.70 | 1.50 | 1.00 | 1.80 | 200.0 |

### Synthesis 5: LPEI/PPO/Bifunctional and Monofunctional Silicone Copolymer

Copolymer samples D1 was produced from starting materials as listed in Table 4. For the sample, LPEI, PPO, and three different types of PDMS, as described in Synthesis 4, were mixed together. The components were combined with IPA in a thick-walled glass flask that was heated to 150° C. and kept at that temperature for 18 hours. The resulting polymer solution was then concentrated to 20% (w/v) using a Roto-Vap.

TABLE 4

| | Si5K | Si1KDi | Si7KDi | PPO | LPEI | IPA |
|---|---|---|---|---|---|---|
| Sample | (g) | (g) | (g) | (g) | (g) | (mL) |
| D1 | 2.89 | 3.80 | 1.70 | 0.40 | 1.80 | 353.0 |

### Application of Samples to Hair

The samples of 20% polymer/IPA solutions were each diluted with deionized water to yield a 0.5% solution and a 0.15% solution (a solution of each concentration was made for each polymer sample). Swatches of hair were dipped into each of the polymer solutions for 5 minutes followed by a 1 minute rinse in water. The samples were then dried with a hair dryer. Half of the hair swatches were wetted and half were kept dry so that the comb-ability (which relates to the hair having a conditioned effect) could be tested in both the wet and dry states. Comb-ability subjectively accesses how smoothly a comb can be run through the swatch of hair. Similar effects were seen at each concentration. The test was repeated in its entirety with the addition of three shampoo wash steps after polymer treatment and rinsing to see the effect of washing on the substantivity of the polymer.

All of the swatches treated using polymer solutions from sample sets A and B had a lesser conditioning effect than those treated from sample sets C and D, but the effect did not lessen with washing. Sample B was more readily dispersible in water than sample A.

Swatches treated using polymer solutions in sample sets C and D all showed an excellent conditioning effect. All except for Sample C2 kept this conditioning effect after washing and showed no decrease in comb-ability. Sample C2 still had some conditioning effect after washing but it was significantly decreased.

While the present invention has been described in terms of specific methods, structures, and devices it is understood that variations and modifications will occur to those skilled in the art upon consideration of the present application. As well, the features illustrated or described in connection with one embodiment may be combined with the features of other embodiments. For example, particular copolymer portions in one embodiment can be combined with one or more copolymer portions of another embodiment. Such modifications and variations are intended to be included within the scope of the present invention. Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

What is claimed is:

1. A hair treatment composition for imparting a conditioning effect to hair, comprising:
a copolymer suitable for use in treating hair comprising:
(i) a plurality of aliphatic amine polymer segments, each segment including a plurality of repeat units, each unit including at least one amine group; and
(ii) a first plurality of silicone segments, each segment being attached to at least two amine groups, each amine group being a part of a distinct aliphatic amine polymer segment.

2. The composition of claim 1, wherein the plurality of aliphatic amine polymer segments include at least one of polyalkyleneimine, polyvinylamine, polyallylamine, polydiallylamine, and copolymers thereof.

3-13. (canceled)

14. The composition of claim 1, wherein the first plurality of silicone segments have an average molecular weight between about 500 Daltons and about 10,000 Daltons.

15. The composition of claim 1, further comprising:
a plurality of softening segments for imparting softness to hair when the copolymer is applied to hair, wherein each of the plurality of softening segments is attached to only one aliphatic amine polymer segment.

16-19. (canceled)

20. A hair treatment composition for imparting a conditioning effect to hair, comprising:
a copolymer suitable for use in treating hair comprising:
(i) a plurality of polycationic segments, each segment including a plurality of repeat units, each unit including at least one amine group;
(ii) a first plurality of silicone segments, each silicone segment being attached to at least one amine group of any of the plurality of polycationic segments; and
(iii) a plurality of hydrophilic segments, each hydrophilic segment being attached to at least one amine group of any of the plurality of polycationic segments.

21. The composition of claim 20, wherein each of the first plurality of silicone segments is attached to at least two amine groups, each amine group being part of a distinct polycationic segment.

22. The composition of claim 20, wherein the plurality of polycationic segments include aliphatic amine polymer segments.

23-34. (canceled)

35. The composition of claim 20, wherein the composition includes a shampoo agent for cleaning hair.

36-52. (canceled)

53. A method of treating hair, comprising:
applying a copolymer to the hair to impart a conditioning effect, the copolymer comprising:
(i) a plurality of polycationic segments, each segment including a plurality of repeat units, each unit including at least one amine group; and
(ii) a plurality of silicone segments, each of the segments being attached to at least one amine group of one polycationic segment.

54. The method of claim 53, wherein the copolymer further comprises:
a plurality of hydrophilic segments for improving water solubility or water dispersibility, each hydrophilic segment being attached to at least one amine group of one polycationic segment.

55. The method of claim 53, wherein the copolymer is not covalently bonded to the hair.

56. The method of claim 53, wherein the step of applying the copolymer comprises:
forming a polymer mixture with the copolymer; and
contacting the polymer mixture with the hair.

57. The method of claim 56, wherein the polymer solution comprises a non-aqueous solvent.

58. The method of claim 56, wherein the polymer mixture comprises an aqueous solvent.

59. The method of claim 53, wherein the step of applying the copolymer comprises:
applying a plurality of polycations to the hair, the polycations having a plurality of repeat units, each unit including at least one amine group;
introducing a mixture comprising silicone polymers, each silicone polymer having at least one functionality for reacting with an amine group of a polycation; and
reacting a silicone polymer with an amine group of a polycation to form the copolymer on the hair.

60. The method of claim 59, wherein the step of applying the copolymer further comprises:
reacting a hydrophilic polymer with another amine group of any of the plurality of polycations to attach the hydrophilic polymer thereto.

61. The method of claim 59, wherein the step of applying the plurality of polycations includes applying a plurality of branched polycations onto the hair.

62. The method of claim 60, wherein the step of reacting the silicone polymer includes at least one of heating the silicone polymers and heating the polycations.

63. The method of claim 53, wherein the method imparts a conditioning effect to the hair.

64. The composition of claim 1, wherein the composition includes a shampoo agent for cleaning hair.
